SEQUENCE LISTING (19 sequences)

SEQ ID NO: 1 - DNA, 489 bp, Gallus gallus
gcgcccgccttccgctacacccgctcacagtccttcgacatcttcgacatcaaccagaagtgcttcgtgctggagtcacccacccagctggtggccctgcacctccaggggccctcctccagccagaaagtgaggctcaacattgcgctgtaccggccccgaggcccacggggcagcgctggaactgggcagatgccagtggcactgggcatcaagggctacaagctctacatgtcgtgtgtgatgagcggcaccgagcccacactgcagctggaggaagccgacgtcatgcgggacatcgacagcgtcgagctgacccgcttcatcttctaccgcctggacagcccgactgagggcaccacgcgcttcgagtcggccgccttccccgggtggttcatctgcacctccctgcagccccggcagcccgtgggcatcaccaaccaacccgaccaggtcaacatcgccacctacaagctaagtgggcgctga

SEQ ID NO: 2 - DNA, 489 bp, artificial sequence
  synthetic sequence
gcgcccgccttccgctacacccgctcacagtccttcgacatcttcgacatcaacgcgaagtgcttcgtgctggagtcacccacccagctggtggccctgcacctccaggggccctcctccagccagaaagtgaggctcaacattgcgctgtaccggccccgaggcccacggggcagcgctggaactgggcagatgccagtggcactgggcatcaagggctacaagctctacatgtcgtgtgtgatgagcggcaccgagcccacactgcagctggaggaagccgacgtcatgcgggacatcgacagcgtcgagctgacccgcttcatcttctaccgcctggacagcccgactgagggcaccacgcgcttcgagtcggccgccttccccgggtggttcatctgcacctccctgcagccccggcagcccgtgggcatcaccaaccaacccgaccaggtcaacatcgccacctacaagctaagtgggcgctga

SEQ ID NO: 3 - DNA, 489 bp, artificial sequence
  synthetic sequence
gcgcccgccttccgctacacccgctcacagtccttcgacatcttcgacatcaaccagaagtgcttcgtgctggagtcacccacccagctggtggccctgcacctccaggggccctcctccagccagaaagtgaggctcaacattgcgctgtaccggccccgaggcccacggggcagcgctggaactgggcagatgccagtggcactgggcatcaagggctacaagctctacatgtcgtgtgtgatgagcggcaccgagcccacactgcagctggaggaagccgacgtcatgcgggacatcgacagcgtcgagctgacccgcttcatcttctaccgcctggacagcccgactgagggcaccacgcgcttcgagtcggccgccttccccgggtggttcatctgcacctccctgcagcccgcgcagcccgtgggcatcaccaaccaacccgaccaggtcaacatcgccacctacaagctaagtgggcgctga

SEQ ID NO: 4 - DNA, 32 bp, artificial sequence
  synthetic primer
cgacatcttcgacatcaacgcgaagtgcttcg

SEQ ID NO: 5 - DNA, 31 bp, artificial sequence
  synthetic primer
ctccagcacgaagcacttcgcgttgatgtcg

SEQ ID NO: 6 - DNA, 29 bp, artificial sequence
  synthetic primer
cacctccctgcagcccgcgcagcccgtgg

SEQ ID NO: 7 - DNA, 30 bp, artificial sequence
  synthetic primer
gtgatgcccacgggctgcgcgggctgcagg

SEQ ID NO: 8 - DNA, 22 bp, artificial sequence
  synthetic primer
ggatccgcccgccttccgctac

SEQ ID NO: 9 - DNA, 24 bp, artificial sequence
  synthetic primer
ctcgagtcagcgcccacttagctt

SEQ ID NO: 10 - DNA, 75 bp, artificial sequence
  synthetic sequence
gcgcccgccttccgctacacccgctcacagtccttcgacatcttcgacatcaacgcgaagtgcttcgtgctggag

SEQ ID NO: 11 - DNA, 454 bp, artificial sequence
  synthetic sequence
cgacatcttcgacatcaacgcgaagtgcttcgtgctggagtcacccacccagctggtggccctgcacctccaggggccctcctccagccagaaagtgaggctcaacattgcgctgtaccggccccgaggcccacggggcagcgctggaactgggcagatgccagtggcactgggcatcaagggctacaagctctacatgtcgtgtgtgatgagcggcaccgagcccacactgcagctggaggaagccgacgtcatgcgggacatcgacagcgtcgagctgacccgcttcatcttctaccgcctggacagcccgactgagggcaccacgcgcttcgagtcggccgccttccccgggtggttcatctgcacctccctgcagccccggcagcccgtgggcatcaccaaccaacccgaccaggtcaacatcgccacctacaagctaagtgggcgctga

SEQ ID NO: 12 - DNA, 436 bp, artificial sequence
  synthetic sequence
cgcccgccttccgctacacccgctcacagtccttcgacatcttcgacatcaaccagaagtgcttcgtgctggagtcacccacccagctggtggccctgcacctccaggggccctcctccagccagaaagtgaggctcaacattgcgctgtaccggccccgaggcccacggggcagcgctggaactgggcagatgccagtggcactgggcatcaagggctacaagctctacatgtcgtgtgtgatgagcggcaccgagcccacactgcagctggaggaagccgacgtcatgcgggacatcgacagcgtcgagctgacccgcttcatcttctaccgcctggacagcccgactgagggcaccacgcgcttcgagtcggccgccttccccgggtggttcatctgcacctccctgcagcccgcgcagcccgtgggcatcac

SEQ ID NO: 13 - DNA, 88 bp, artificial sequence
  synthetic sequence
cacctccctgcagcccgcgcagcccgtgggcatcaccaaccaacccgaccaggtcaacatcgccacctacaagctaagtgggcgctga

SEQ ID NO: 14 - DNA, 489 bp, Gallus gallus
gcgcccgccttccgctacgcccgctcacagtccttcgacatcttcgacatcaaccagaagtgcttcgtgctggagtcacccacccagctggtggccctgcacctccaggggccctcctccagccagaaagtgaggctcaacattgcgctgtaccggccccgaggcccacggggcagcgctggaactgggcagatgccagtggcactgggcatcaagggctacaagctctacatgtcgtgtgtgatgagcggcaccgagcccacactgcagctggaggaagccgacgtcatgcgggacatcgacagcgtcgagctgacccgcttcatcttctaccgcctggacagcccgactgagggcaccacgcgcttcgagtcggccgccttccccgggtggttcatctgcacctccctgcagccccggcagcccgtgggcatcaccaaccaacccgaccaggtcaacatcgccacctacaagctaagtgggcgctga

SEQ ID NO: 15 - DNA, 27 bp, Artificial Sequence
  synthetic primer
cccgccttccgctacgcccgctcacag

SEQ ID NO: 16 - DNA, 28 bp, Artificial Sequence
  synthetic primer
gaaggactgtgagcgggcgtagcggaag

SEQ ID NO: 17 - DNA, 489 bp, Gallus gallus
gcgcccgccttccgctacacccgctcacagtccttcgacatcttcgacatcaaccagaagtgcttcgtgctggagtcacccacccagctggtggccctgcacctccaggggccctcctccagccagaaagtgaggctcaacattgcgctgtaccggccccgaggcccacggggcagcgctggaactgggcagatgccagtggcactgggcatcaagggctacaagctctacatgtcgtgtgtgatgagcggcaccgagcccacactgcagctggaggaagccgacgtcatgcgggacatcgacagcgtcgagctgacccgcttcatcttctaccgcctggacagcccgactcggggcaccacgcgcttcgagtcggccgccttccccgggtggttcatctgcacctccctgcagccccggcagcccgtgggcatcaccaaccaacccgaccaggtcaacatcgccacctacaagctaagtgggcgctga

SEQ ID NO: 18 - DNA, 28 bp, Artificial Sequence
  synthetic primer
ctggacagcccgactcggggcaccacgc

SEQ ID NO: 19 - DNA, 28 bp, Artificial Sequence
  synthetic primer
gaagcgcgtggtgccccgagtcgggctg

(19) United States
(12) Patent Application Publication
(10) Pub. No.: US 2019/0276512 A1
(43) Pub. Date: Sep. 12, 2019

(54) RECOMBINANT CHICKEN INTERLEUKIN-1 BETA PROTEIN FOR PRODUCING ANTIBODY EARLY AND RETAINING FOR A LONGER PERIOD OF TIME AND APPLICATION THEREOF

(71) Applicant: National Tsing Hua University, Hsinchu (TW)

(72) Inventors: Hsien-Sheng Yin, Hsinchu City (TW); Wen-Ting Chen, Hsinchu City (TW); Hsin-Yu Yang, Hsinchu City (TW); Chih-Yu Lin, Hsinchu City (TW)

(21) Appl. No.: 16/424,693

(22) Filed: May 29, 2019

Related U.S. Application Data
(63) Continuation-in-part of application No. 15/153,704, filed on May 12, 2016.

(30) Foreign Application Priority Data
Mar. 23, 2016 (TW) 105109016

(51) Int. Cl. C07K 14/545; A61K 39/39; A61K 39/17; C12N 7/00; A61P 31/14

(52) U.S. Cl. CPC C07K 14/545; A61K 39/39; A61K 2039/55527; C12N 7/00; A61P 31/14; A61K 39/17

(57) ABSTRACT

The present invention provides a recombinant chicken interleukin-1β protein for producing antibody early and retaining for a longer period of time, which has a sequence of SEQ ID NO:2 or SEQ ID NO:3. The recombinant chicken interleukin-1β protein is created by using point mutation in a genetic engineering method; it can significantly improve the original vaccine efficacy to enhance antibody responses, produce antibody one week earlier and extend the protective effect until chickens sold off. Therefore, the recombinant chicken interleukin-1β protein of the present invention can produce significant higher antibody responses than the with-type chicken interleukin-1β protein, it helps to develop avian interleukin-1β vaccine adjuvant and uses in medical application and livestock production.

### CROSS-REFERENCE TO RELATED APPLICATION

This application is a continuation-in-part of U.S. patent application Ser. No. 15/153,704, filed on May 12, 2016, which claims the priority benefit of Taiwan patent application No. 105109016 filed on Mar. 23, 2016. The entirety of each of the above-mentioned patent applications is hereby incorporated by reference herein and made a part of this specification.

### BACKGROUND OF THE INVENTION

### 1. Field of the Invention

The present invention relates to a recombinant chicken interleukin-1β protein, and in particular, to a recombinant chicken interleukin-1β protein for producing antibody early and retaining for a longer period of time

### 2. The Prior Arts

The poultry industry in global continues to show impressive growth year by year. Therefore, the poultry farm in developed areas use a high density confinement rearing method to increase breeding density, which causes an urgent problem, government agency should spend more effort into biosecurity and disease prevention. In recent years, some countries have been faced the difficult challenges of a dramatic increase in the incidence of infectious disease outbreaks, some result from poor hygiene and management, but most of which result from a highly aggressive strain of virus, such as avian infectious bronchitis, infectious bursal disease, Newcastle disease and avian influenza, etc. In 2005, there were outbreaks of avian influenza in southern Taiwan, which not only caused a lot of deaths in ducks and geese, but also endangered human lives and resulted in huge economic impact and financial crisis. The best way to prevent infection with avian influenza is to avoid sources of exposure whenever possible. Most bird infections with avian influenza in poultry farms will be culled to avoid the viruses spreading. However, in high-density poultry farms, once the high-pathogenic avian influenza outbreaks, the speed of culling the infected birds may be less than that of the high-pathogenic avian influenza spread. Therefore, administrating a high-quality vaccine or vaccine adjuvant is the most effective way to prevent infection and severe outcomes caused by influenza viruses. And it needs to step up the preventive measures against avian influenza.

For animal vaccines, there are five poultry vaccines against Newcastle disease, avian infectious bronchitis, infectious bursal disease and fowl pox, which occupies 70 percent in vaccine testing, one third of the live attenuated fowl pox virus vaccines is produced in Taiwan, nearly 90 percent of the remaining vaccines are imported from foreign countries. Moreover, the adjuvant in a high titer avian vaccine is only obtained from foreign countries, which leads to higher prices and the lack of market competitiveness.

Recently, two main known vaccine adjuvants, aluminum-based and oil-based, are used in global livestock. The adverse effect of both vaccine adjuvants is chemical-makeup, wherein the aluminum-based adjuvant cannot enhance the specificity of the Th1 cell immune response. Oil-based adjuvant can trigger immune system response, but it may result in local inflammation and granulomatous reactions at the site of injection, chronic inflammation, skin ulceration, local abscess or tissue sloughing, diffuse systemic granulomas, and it is also unable to enhance the production of antibodies.

### SUMMARY OF THE INVENTION

As such, the present invention provides a recombinant chicken interleukin-1β protein for producing antibody early and retaining for a longer period of time, which is created by point mutation in a genetic engineering method. The recombinant chicken interleukin-1β protein of the present invention can significantly improve the original vaccine efficacy to enhance antibody responses, to produce antibody one week earlier and to extend the protective effect until chickens sold off, and can produce significant higher antibody responses than the with-type chicken interleukin-1β protein.

A primary objective of the present invention is to provide a recombinant chicken interleukin-1β protein, which is encoded by the nucleotide sequence of SEQ ID NO:2.

Another objective of the present invention is to provide a method of producing antibody early and retaining for a longer period of time by using the recombinant chicken interleukin-1β protein.

A further objective of the present invention is to provide a method of producing antibody early and retaining for a longer period of time by using a vaccine supplemented with the recombinant chicken interleukin-1β protein.

According to an embodiment of the present invention, a time for producing antibody is one week earlier

According to an embodiment of the present invention, the longer period of time is at least four weeks to extend the protective effect, and a booster is not given.

According to an embodiment of the present invention, the recombinant chicken interleukin-1β protein is a component of a vaccine.

According to an embodiment of the present invention, the vaccine is an inactivated vaccine or an attenuated vaccine.

According to an embodiment of the present invention, the recombinant chicken interleukin-1β protein is a component of a vaccine adjuvant.

Accordingly, the recombinant chicken interleukin-1β protein of the present invention as a biological adjuvant directly use with an inactivated or activated avian vaccine used in the veterinary vaccines market, it can significantly improve the original vaccine efficacy to enhance antibody responses, and to produce neutralizing antibody against virus earlier. The recombinant chicken interleukin-1β protein of the present invention helps to develop avian interleukin-1β vaccine adjuvant and uses in medical application and livestock production.

### BRIEF DESCRIPTION OF THE DRAWINGS

The present invention will be apparent to those skilled in the art by reading the following detailed description of a preferred embodiment thereof, with reference to the attached drawings, in which:

FIG. 1 is a flow chart of constructing the recombinant chicken interleukin-1β (IL-1β) protein of the present invention;

FIG. 2 is 12% sodium dodecyl sulfate polyacrylamide gel electrophoresis (SDS PAGE) of the recombinant chicken interleukin-1β (IL-1β) protein of the present invention after clone, recombinant protein expression and purification, ChIL-1β mutant 1 is indicated the recombinant chicken interleukin-1β protein having the first mutation and ChIL-1β mutant 2 is indicated the recombinant chicken interleukin-1β protein having the second mutation;

FIG. 3 shows that the plasma cortisol levels in chickens administrated the recombinant chicken interleukin-1β protein, PBS is indicated phosphate-buffered saline; HmIL-1β is indicated human interleukin-1β, ChIL-1β is indicated wild-type chicken interleukin-1β, ChIL-1β mutant 1 is indicated the recombinant chicken interleukin-1β protein having the first mutation and ChIL-1β mutant 2 is indicated the recombinant chicken interleukin-1β protein having the second mutation;

FIG. 4 shows HI antibody titers of the chickens administrated Newcastle disease live attenuated vaccine supplemented with the recombinant chicken interleukin-1β protein via eye drop using hemagglutination inhibition test, ChIL-1β is indicated wild-type chicken interleukin-1β, ChIL-1β MU1 is indicated the recombinant chicken interleukin-1β protein having the first mutation, ChIL-1β MU2 is indicated the recombinant chicken interleukin-1β protein having the second mutation, control is indicated PBS; pre is indicated before eye drop vaccination, PRI is indicated a primary dose, a booster is indicated a second dose;

FIG. 5 shows the antibody level of the chickens administrated ND vaccine supplemented with the recombinant chicken interleukin-1β protein, PBS is indicated phosphate-buffered saline, ChIL-1β is indicated wild-type chicken interleukin-1β, ChIL-1β mutant 1 is indicated the recombinant chicken interleukin-1β protein having the first mutation and ChIL-1β mutant 2 is indicated the recombinant chicken interleukin-1β protein having the second mutation;

FIG. 6 shows immunohistochemistry staining of the chickens administrated ND vaccine supplemented with the recombinant chicken interleukin-1β protein, A is indicated to administrate PBS, B is indicated to administrate vaccine, C is indicated to administrate vaccine supplemented with wild-type interleukin-1β protein (ChIL-1β), D is indicated to administrate vaccine supplemented with the recombinant chicken interleukin-1β protein having the first mutation (ChIL-1β mutant 1) and E is indicated to administrate vaccine supplemented with the recombinant chicken interleukin-1β protein having the second mutation (ChIL-1β mutant 2);

FIG. 7 shows HI antibody titers of the chickens administrated one dose of live attenuated Newcastle disease vaccine supplemented with the recombinant chicken interleukin-1β protein using hemagglutination inhibition test, ChIL-1β is indicated wild-type chicken interleukin-1β, ChIL-1β MU2 is indicated the recombinant chicken interleukin-1β protein having the second mutation, control is indicated PBS; pre is indicated before eye drop vaccination, PRI is indicated a primary dose, 14 is indicated after administrating 2 weeks, 28 is indicated after administrating 4 weeks.

FIG. 8 is an electropherogram of the soluble wild-type chicken IL-1β recombinant protein (ChIL-1β) and mutant chicken IL-1β recombinant proteins (ChIL-1β T7A and ChIL-1β E118R) with high-purity.

FIG. 9 is a data diagram showing the comparison of biological activity among the wild-type chicken IL-1β and mutant chicken IL-1β to induce plasma cortisol in vivo.

FIG. 10 is a data diagram showing the HI antibody titers of anti-NDV antibody in the blood produced by the NDV vaccine (or mixed with the wild-type chicken IL-1β or mutant chicken IL-1β) 0, 1, 2, and 3 weeks after nasal drop vaccination.

### DETAILED DESCRIPTION OF THE PREFERRED EMBODIMENT

The accompanying drawings are included to provide a further understanding of the invention, and are incorporated in and constitute a part of this specification. The drawings illustrate embodiments of the invention and, together with the description, serve to explain the principles of the invention.

The present invention provides a recombinant chicken interleukin-1β protein for producing antibody early and retaining for a longer period of time, which can stimulate cytokine secretion and enhance antibody production. The recombinant chicken interleukin-1β protein is an effective and low cost vaccine adjuvant for outbreaks of avian disease.

Therefore, the present invention constructs two new recombinant chicken interleukin-1β protein from an improved design of the amino acid sequence of wild-type chicken interleukin-1β (IL-1β), which are respectively named as the recombinant chicken interleukin-1β protein having first mutation (ChIL-1β mutant 1) and the recombinant chicken interleukin-1β protein having second mutation (ChIL-1β mutant 2). The present invention uses the recombinant chicken interleukin-1β proteins with a common vaccine in Taiwan and global poultry industry, Newcastle disease (ND), to conduct animal clinical trials. The results show that the recombinant chicken interleukin-1β protein of the present invention can stimulate the immune system to produce antibody for ND vaccine, and the chicken can only receive one dose to continue producing antibodies and to extend the protective effect. The local tissue administrated vaccine or vaccine adjuvant comprising the recombinant chicken interleukin-1β protein not only can produce secretory immunoglobulin A (IgA) but also maintain a high level of immunoglobulin G (IgG) in the blood. In addition, the vaccine comprising the recombinant chicken interleukin-1β protein can have positive synergistic effect on high IgG antibody production rates to extend the protective effect until chickens sold off. Therefore, the recombinant chicken interleukin-1β protein of the present invention helps to develop avian vaccine adjuvant and uses in medical application and livestock production.

### Example 1

### Construct the Recombinant Chicken Interleukin-1B Protein

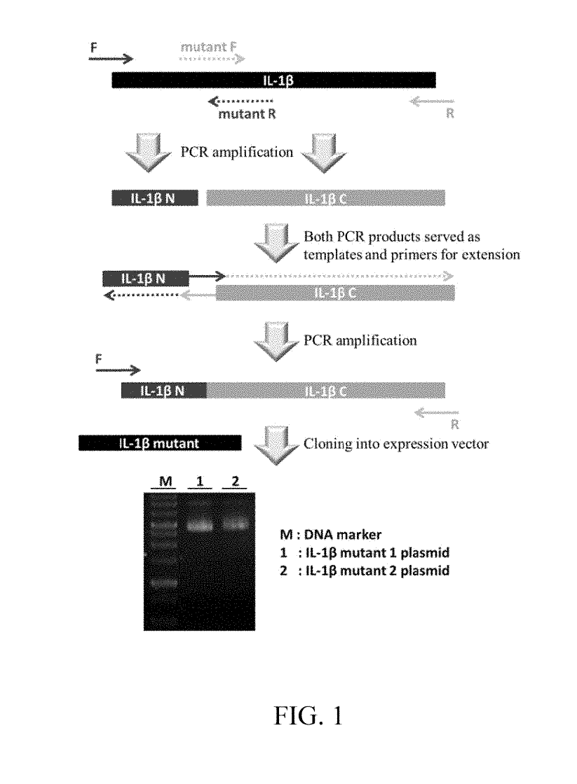

The flow chart of constructing the circular permutation interleukin-1β (CP IL-1β) in one embodiment of the present invention as shown in FIG. 1. The present invention designs a wild-type chicken interleukin-1β sequence (SEQ ID NO:1) to have a point mutation, which is obtained from predicting an amino acid position that may affect biological activity by using a 3D structure of wild-type chicken interleukin-1β and its receptors, the point mutations are respectively Q19A and R140A. Therefore, the present mutation constructs the first mutation of the recombinant chicken interleukin-1β sequence having Q19A (SEQ ID NO:2) and the second mutation of the recombinant chicken interleukin-1β sequence having R140A (SEQ ID NO:3).

First, designing the forward and reverse primers comprising the point mutation for the two point mutation sequences of chicken interleukin-1β protein (SEQ ID NO:2 and SEQ ID NO: 3), wherein Q19A forward and reverse primers of the first mutation sequence (SEQ ID NO:2) respectively are SEQ ID NO: 4 and SEQ ID NO: 5; R140A forward and reverse primers of the second mutation sequence (SEQ ID NO:3) respectively are SEQ ID NO:6 and SEQ ID NO:7. And, using polymerase chain reaction to amplify wild-type chicken interleukin-1β sequence (SEQ ID NO:1) for the mutation site, wherein wild-type chicken interleukin-1β sequence is as a template (SEQ ID NO:1), the forward primer of wild-type chicken interleukin-1β (SEQ ID NO:8) and Q19A reverse primer (SEQ ID NO: 5) amplify Q19A mutation site to obtain the first fragment of the first mutation sequence (SEQ ID NO: 10), Q19A forward primer (SEQ ID NO:4) and the reverse primer of wild-type chicken interleukin-1β (SEQ ID NO: 9) amplify Q19A mutation site to obtain the second fragment of the first mutation sequence (SEQ ID NO: 11); and the forward primer of wild-type chicken interleukin-1β (SEQ ID NO:8) and R140A reverse primer (SEQ ID NO: 7) amplify R140A mutation site to obtain the first fragment of the second mutation sequence (SEQ ID NO: 12), R140A forward primer (SEQ ID NO:6) and the reverse primer of wild-type chicken interleukin-1β (SEQ ID NO: 9) amplify R140A mutation site to obtain the second fragment of the second mutation sequence (SEQ ID NO: 13). Then, the first (SEQ ID NO: 10) and second (SEQ ID NO: 11) fragments of the first mutation sequence serve as templates and primers for extension to obtain the first mutation of the recombinant chicken interleukin-1β sequence having Q19A mutation site (SEQ ID NO:2); and the first (SEQ ID NO: 12) and second (SEQ ID NO: 13) fragments of the second mutation sequence serve as templates and primers for extension to obtain the second mutation of the recombinant chicken interleukin-1β sequence having R140A mutation site (SEQ ID NO:3).

Furthermore, amplifying the first mutation of the recombinant chicken interleukin-1β sequence having Q19A (SEQ ID NO:2) as template using the forward (SEQ ID NO:8) and reverse (SEQ ID NO:9) primers of wild-type chicken interleukin-1β; and amplifying the second mutation of the recombinant chicken interleukin-1β sequence having R140A mutation site (SEQ ID NO:3) as template using the forward (SEQ ID NO:8) and reverse (SEQ ID NO:9) primers of wild-type chicken interleukin-1β.

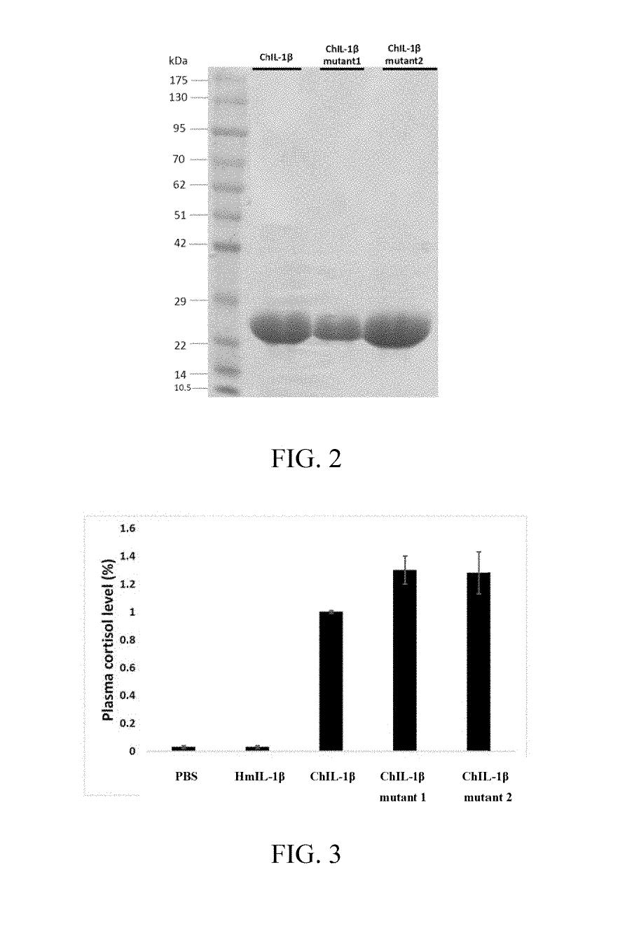

Finally, cloning, expressing and purifying wild-type chicken interleukin-1β sequence (SEQ ID NO: 1), the first mutation of the recombinant chicken interleukin-1β sequence (SEQ ID NO: 2), and the second mutation of the recombinant chicken interleukin-1β sequence (SEQ ID NO: 3). As shown in FIG. 2, the molecular weight of wild-type chicken IL-1β (ChIL-1β), the recombinant chicken interleukin-1β protein having the first mutation (ChIL-1β mutant 1) and the recombinant chicken interleukin-1β protein having the second mutation (ChIL-1β mutant 2) is 23.6 kDa by 12% sodium dodecyl sulfate polyacrylamide gel electrophoresis (SDS PAGE).

### Example 2

### Bioactivity Assay of the Recombinant Chicken Interleukin-1β

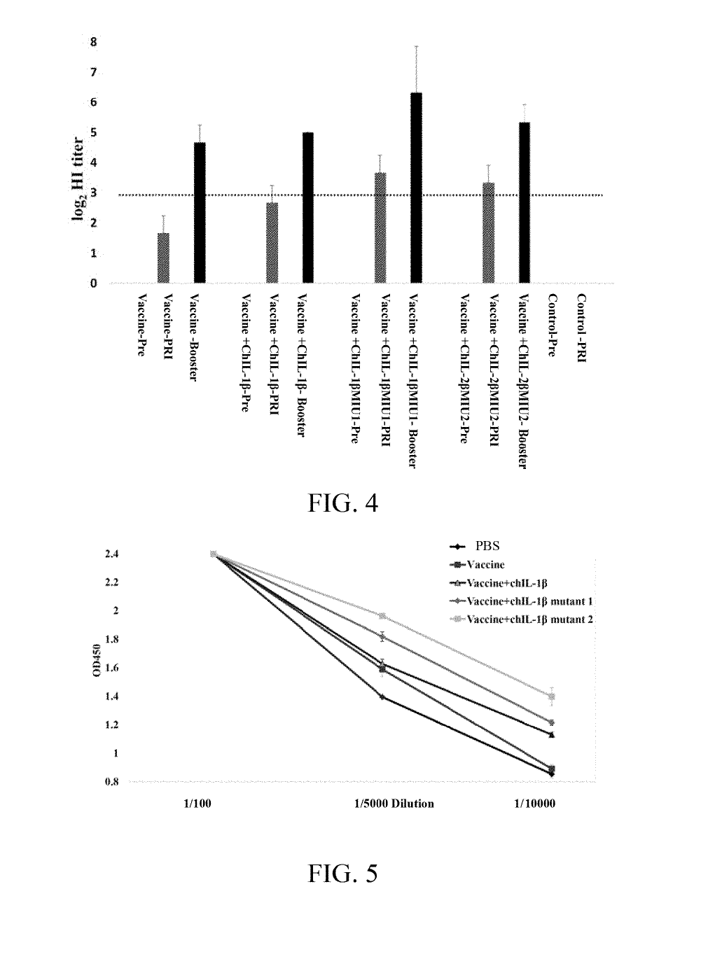

To determine the in vivo activity of the recombinant chicken interleukin-1β, the present invention detects the plasma cortisol level after the recombinant chicken interleukin-1β proteins having the first (ChIL-1β mutant 1) and the second (ChIL-1β mutant 2) mutation are respectively injected into the wing vein of specific pathogen free (SPF) chicken. As shown in FIG. 3, the plasma cortisol levels in chickens are significantly enhanced by intravenous injections of the recombinant chicken interleukin-1β protein having the first (ChIL-1β mutant 1) or the second (ChIL-1β mutant 2) mutation, but the same result is not found in the chicken injected phosphate-buffered saline (PBS). Therefore, the recombinant chicken interleukin-1β protein of the present invention has more bio activity than wild-type chicken interleukin-1β protein. The result indicates that the recombinant chicken interleukin-1β protein has great benefit for vaccine adjuvant.

### Example 3

### Hemagglutination Inhibition Test (HI Test) of the Recombinant Chicken Interleukin-1β

To determine vaccine adjuvant effect of the recombinant chicken interleukin-1β protein, the present invention administrates Newcastle disease (ND) live attenuated vaccine or which supplemented with the recombinant chicken interleukin-1β protein to specific pathogen free (SPF) chickens via eye drop, the chickens at one week of age is administrated primary (PRI) dose, the chickens at three weeks of age is administrated booster dose. The present invention draws blood samples from the wing vein of the chickens at two and four weeks of age to evaluate the immune response to Newcastle disease vaccine by measuring HI antibody titers using hemagglutination inhibition test.

As shown in FIG. 4, the chickens only administrated Newcastle disease vaccine (vaccine-PRI) cannot produce enough antibody against Newcastle disease at two weeks of age. Only a few of the chickens administrated Newcastle disease vaccine supplemented with wild-type chicken interleukin-1β protein (vaccine+IL-1β) can produce enough antibodies against Newcastle disease at two weeks of age. All the chickens administrated Newcastle disease vaccine supplemented with the recombinant chicken interleukin-1β protein having the first (vaccine+ChIL-1β MU1-PRI) or the second mutation (vaccine+ChIL-1β MU2-PRI) can produce enough antibodies against Newcastle disease at two weeks of age. These results show that the recombinant chicken interleukin-1β protein can be a vaccine adjuvant to promote the host producing antibody earlier and extending the protective titers.

### Example 4

### Antibody Levels Produced by the Recombinant Chicken Interleukin-1β

To evaluate the humoral immunity response produced by the host administrated the recombinant chicken interleukin-1β as vaccine adjuvant, the present invention performs a 1:100 dilution of the serum obtained from EXAMPLE 3 to measure the quantity of avian IgG using enzyme-linked immunosorbent assay (ELISA).

As shown in FIG. 5 and Table 1, the IgG level in each sample is larger than the maximum of standard curve, and the serum is made dilution of 1:5000 and 1:10000. These results show that the IgG level of the host administrated ND vaccine supplemented with the recombinant chicken interleukin-1β is more than the host only administrated a ND vaccine or a ND vaccine supplement with wild-type chicken interleukin-1β, and the host only administrated PBS via eye drop is no IgG level in the blood. Therefore, these results validate that the recombinant chicken interleukin-1β can be a vaccine adjuvant to help ND vaccine elevating IgG level in the serum, it can generate immune responses against ND disease virus and other microorganisms that cause disease can be bacteria or virus.

TABLE 1
the serum IgG level of the host administrated ND live
attenuated vaccine and/or the recombinant chicken interleukin-
1β of the present invention via eye drop

| | Dilution Range, ng/ml | | |
|---|---|---|---|
| Sample | 1/100 | 1/5000 | 1/10000 |
| Mock | 200.399 | 48.285 | 25.015 |
| Vaccine | 201.578 | 59.678 | 26.371 |
| Vaccine + chIL-1β | 203.123 | 62.276 | 35.772 |
| Vaccine + chIL-1β mutant 1 | 202.690 | 77.835 | 39.521 |
| Vaccine + chIL-1β mutant 2 | 201.446 | 95.087 | 48.417 |

### Example 5

### The Immunohistochemistry Staining of the Recombinant Chicken Interleukin-113

To confirm the IgA distribution secreted by mucosal immune system in the nasal cavity of the host after administrating ND live attenuated vaccine supplemented with the recombinant chicken interleukin-1β protein via nose drop. The nasal cavity tissues are cut into slices three weeks after vaccination and stained using immunohistochemistry.

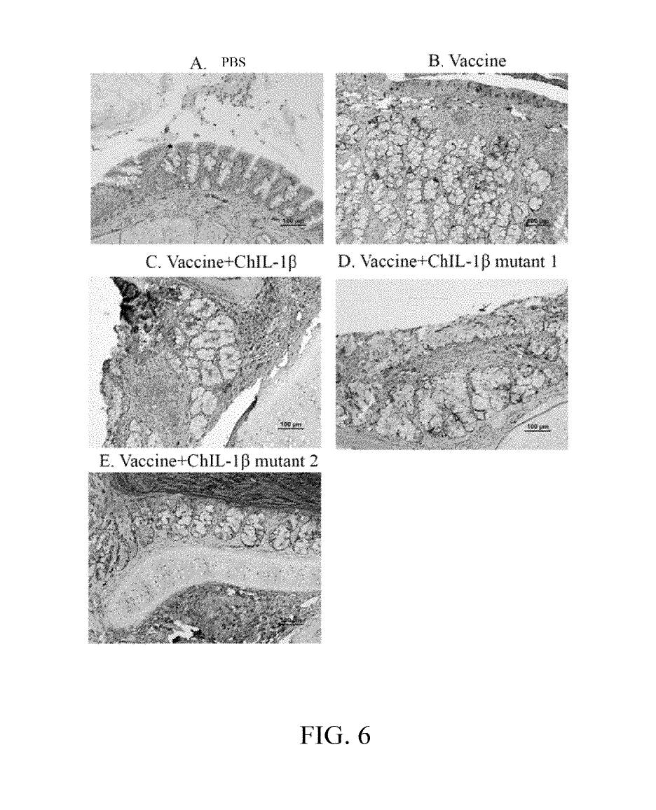

As show in FIG. 6, the vaccine supplemented with the recombinant chicken interleukin-1β protein having the first (ChIL-1β mutant 1) or the second mutation (ChIL-1β mutant 2) can stimulate lymphatic tissue, nasal epithelial cells and around glandular cells to produce a large amount of IgA distribution (FIGS. 6D and 6E), the vaccine supplemented with wild-type chicken interleukin-1β protein can stimulate nasal epithelial cells and glandular cells to produce a moderate amount of IgA distribution (FIG. 6C). However, only the vaccine administrated via nose drop can stimulate nasal epithelial cells and glandular cells to produce a few of IgA distribution (FIG. 6B), only PBS administrated via nose drop cannot stimulate glandular cells and lymphatic tissue in nasal cavity to produce any IgA (FIG. 6A). Therefore, these results validate that the recombinant chicken interleukin-1β can be a vaccine adjuvant to stimulate mucosal immune system producing a large amount of secretory IgA antibodies.

### Example 6

### The Recombinant Chicken Interleukin-1β has the Capability of Producing Antibody Earlier and Extending the Protective Effect

To test the protective effect of only administrating host one dose of ND live attenuated vaccine supplemented with the recombinant chicken interleukin-1β protein. The present invention follows EXAMPLE 3 protocol to administrate host at one week of age one dose of ND vaccine supplemented with the vaccine adjuvant of the recombinant chicken interleukin-1β protein having the second mutation (ChIL-1β mutant 2), and draws blood samples of host at two and four weeks of age to evaluate the immune response by measuring HI antibody titers using hemagglutination inhibition test.

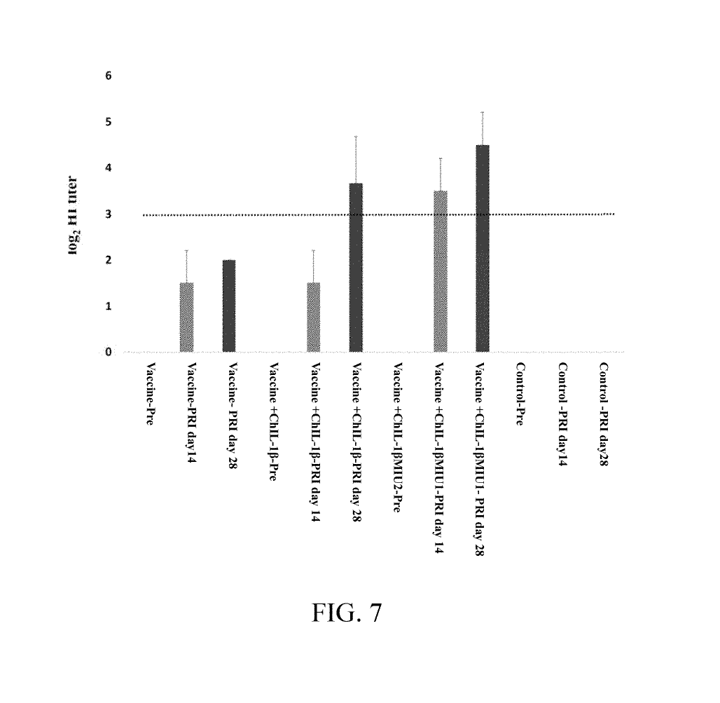

As shown in FIG. 7, the host administrated the ND vaccine adjuvant of the recombinant chicken interleukin-1β protein having the second mutation (ChIL-1β mutant 2) can produce enough antibodies against ND virus at two weeks of age and extend the protective effect at four weeks of age. But the host administrated one dose of the ND vaccine supplemented with wild-type chicken interleukin-1β protein cannot produce enough antibodies against ND virus until the host at four weeks of age. Therefore, the recombinant chicken interleukin-1β protein of the present invention as a vaccine adjuvant supplementing with a vaccine is only administrated a host one dose to extend the protective effect and not to be weakened over time. The recombinant chicken interleukin-1β protein can save booster cost.

### Comparative Example

Not all Mutant Chicken IL-1β Proteins have Better Vaccine Adjuvant Effects than Those of Wild-Type Chicken IL-1β Proteins

In order to confirm that not all mutant chicken IL-1β proteins, as Q19A and R140A, have better vaccine adjuvant effects than those of wild-type chicken IL-1β proteins, novel mutant chicken IL-1β_T7A and IL-1β_E118R genes are designed. Gene cloning, recombinant protein expression, mutant chicken recombinant protein purification, and in vivo activity analysis were performed. Further, as the ND vaccine adjuvant and mixed vaccine immunized chicken, blood drawing per week for three weeks after immunization was performed to monitor the relative titer of antibodies against ND virus. The results show that the biological activity of mutant chicken IL-1β_T7A is similar to that of wild-type chicken IL-1β, and the effect as an NDV vaccine adjuvant of mutant chicken IL-1β_T7A is similar to that of wild-type chicken IL-1β. More interestingly, mutant chicken IL-1β_E118R not only has almost no biological activity, but it is almost ineffective as an NDV vaccine adjuvant. These results indicate that not all mutant chicken IL-1β proteins that have single point mutation on amino acid have better vaccine adjuvant effects than those of wild-type chicken IL-1β proteins.

First, the genetic recombination cloning method was constructed. The primers required for the amplification of the mutant chicken IL-1β DNA were designed, and submitted to MDBio, Inc. for sequence synthesis (Table 2). These designs are designed to mutate the seventh amino acid residue of threonine (T) into alanine (A) in the amino acid sequence of chicken and the newly constructed mutant gene is called ChIL-1β_T7A (SEQ ID NO: 14). In addition, the 118th amino acid residue of glutamate was replaced by arginine (R), and the newly constructed mutant gene name is called ChIL-1β_E118R (SEQ ID NO: 17).

TABLE 2

| Primer name | Primer sequence |
|---|---|
| Forward primer for | 5′-CCCGCCTTCCGCTACGCCCGCTCAC |
| ChIL-1β_T7A | AG-3′ |
| | (SEQ ID NO: 15) |
| Reverse primer for | 5′-GAAGGACTGTGAGCGGGCGTAGCGG |
| ChIL-1β_T7A | AAG-3′ |
| | (SEQ ID NO: 16) |
| Forward primer for | 5′-CTGGACAGCCCGACTCGGGGCACCA |
| ChIL-1β_E118R | CGC-3′ |
| | (SEQ ID NO: 18) |
| Reverse primer for | 5′-GAAGCGCGTGGTGCCCCGAGTCGGG |
| ChIL-1β_E118R | CTG-3′ |
| | (SEQ ID NO: 19) |

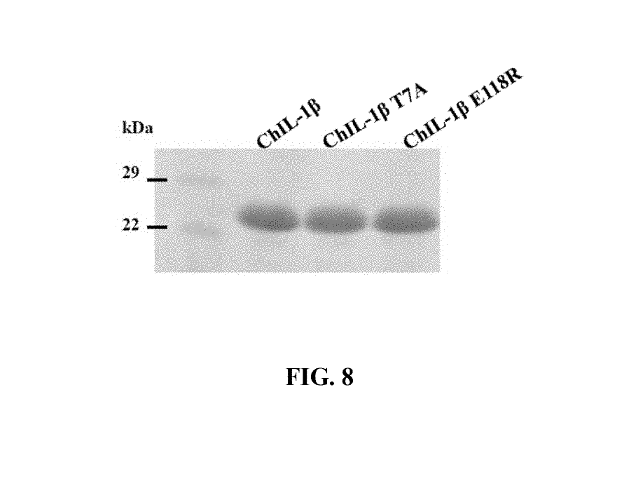

First, wild-type chicken IL-1β (ChIL-1β) and mutant ChIL-1βT7A and E118R, which are newly synthesized and amplified, were genetically selected, and then the correctness of the sequences were detected by DNA sequencing. The E. coli expression system was used to express and purify mutant chicken IL-1β recombinant proteins, and soluble proteins with high-yield and high-purity were successfully obtained. The molecular weight of the mutant chicken IL-1β recombinant proteins is similar to that of the wild-type chicken IL-1β protein, which is about 23.6 kDa (FIG. 8).

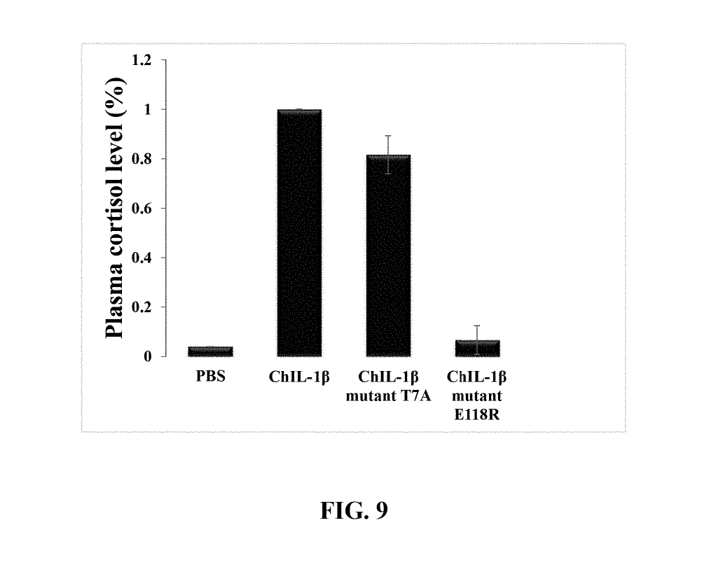

To examine the biological activity of the designed mutant IL-1β in chickens, a plasma cortisol concentration analysis was performed in vivo. Phosphate buffer solution (PBS), the wild-type chicken IL-1β (ChIL-1β) protein, the mutant chicken IL-1β T7A (ChIL-1β mutant T7A) protein, and the mutant chicken IL-1β E118R (ChIL-1β mutant E118R) protein were injected intravenously into the wing vein of specific pathogen free (SPF) chicken, because functional chicken IL-1β induces the chicken central system to produce cortisol to enter the bloodstream to resist IL-1β. After three hours, the blood of the chickens under test was taken, and the relative plasma cortisol levels induced by the wild-type chicken IL-1β protein and mutant chicken IL-1β proteins were measured (FIG. 9).

The result shows that the relative plasma cortisol level induced by ChIL-1β mutant T7A was similar to that of the wild-type chicken IL-1β, and the relative plasma cortisol level induced by ChIL-1β mutant E118R was significantly decreased, almost the same as the cortisol level in the central system induced by PBS in the negative control group. Compared with the relative plasma cortisol level induced by the wild-type chicken IL-1β, the relative plasma cortisol level induced by ChIL-1β mutant T7A and ChIL-1β mutant E118R was 82% and 10%, respectively.

That is, ChIL-1β mutant T7A has almost similar functionality to the wild-type chicken IL-1β, and the functionality is not improved due to point mutation on amino acid residues (threonine to alanine). On the contrary, the functionality is maintained as that of the wild-type chicken IL-1β. Further, ChIL-1β mutant E118R almost loses the functionality of chicken IL-1β, and the ability to induce cortisol production by ChIL-1β mutant E118R is lost as that of PBS in the negative control group. Therefore, not changing any amino acid residue on the wild-type chicken IL-1β can enhance the function of the wild-type chicken IL-1β. ChIL-1β mutant E118R is similar to PBS, but has little ability to induce cortisol (FIG. 9).

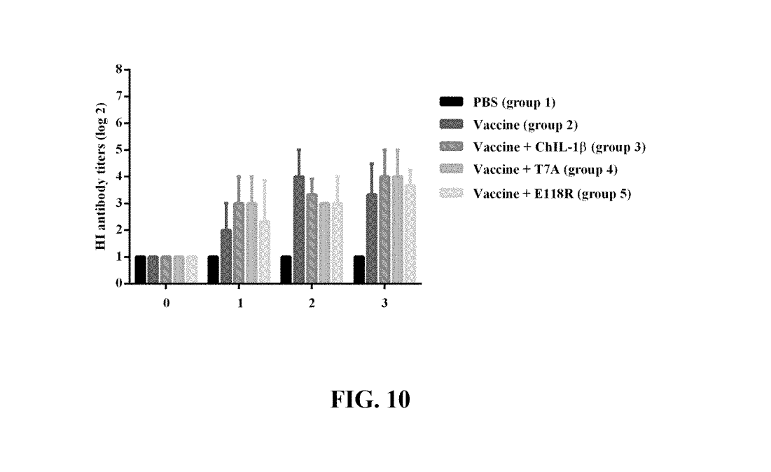

In order to further understand whether chicken IL-1β or mutant chicken IL-1β proteins in which any amino acid residue alteration was constructed (e.g., ChIL-1β mutant T7A and ChIL-1β mutant E118R) can be used as a chicken vaccine adjuvant, chicken Newcastle disease (ND)-live vaccine (VOLVAC ND LaSota MLU, Boehringer Ingelheim Vetmedica, S.A. de C.V., Mexico) alone without chicken IL-1β (group 2), mixed with 1 μg of wild-type chicken IL-1β (group 3), ChIL-1β mutant T7A (group 4), or ChIL-1β mutant E118R (group 5) was vaccinated to one-week-old SPF chicks via nasal drop vaccination once. Another group of chicks as a negative control group were vaccinated with PBS via nasal drop vaccination. 0, 1, 2, and 3 weeks after vaccination, blood was drawn from the chicken wing vein, and these sera were subjected to anti-NDV hemagglutination inhibition (HI) test to detect the HI antibody titers against NDV when the wild-type chicken IL-1β and mutant chicken IL-1β were used as an NDV vaccine adjuvant (FIG. 10).

The result indicates that the HI antibody titers of protective antibody produced by ChIL-1β mutant T7A (group 4) assisted NDV vaccine 1, 2, and 3 weeks after vaccination were similar to those of the wild-type chicken IL-1β (group 3), and the HI antibody titers of protective antibody produced by ChIL-1β mutant E118R (group 5) assisted NDV vaccine 1, 2, and 3 weeks after vaccination were similar to those of the NDV vaccine alone (FIG. 10). These results indicate that mutant chicken IL-1β proteins formed by point mutation on amino acid residues of the wild-type chicken IL-1β do not have better adjuvant effect (such as ChIL-1β mutant T7A) than that of the wild-type chicken IL-1β, and even do not have the ability to be used as an immunological adjuvant (such as ChIL-1β mutant E118R).

The present invention is to create two kinds of the recombinant chicken interleukin-1β protein as vaccine adjuvant using point mutation in a genetic engineering method, it can significantly enhance the capability of producing antibody, produce antibody one week earlier, and extend the protective effect until chicken sold off. Furthermore, the present invention has validated that the recombinant chicken interleukin-1β protein as a vaccine adjuvant using with Newcastle disease (ND) has significant effects on immune response. Also, the cost of the recombinant chicken interleukin-1β protein as a vaccine adjuvant is 0.1 Taiwan Dollar (TWD) for each chicken, it can be more lower cost resulted from mass production to produce market competition. The recombinant chicken interleukin-1β protein has a biological decomposition, and it can store at a room temperature or in the freezer to maintain activity for 4 to 10 days (not precipitation), and store at −20° C. to maintain activity for 6 months (not precipitation). Therefore, the recombinant chicken interleukin-1β protein has good quality, stability, safety and including no side effects.

Accordingly, the recombinant chicken interleukin-1β protein of the present invention as a biological adjuvant directly uses with an inactivated or activated avian vaccine used in the veterinary vaccines market, it can significantly improve the original vaccine efficacy to enhance antibody responses, produce neutralizing antibody against virus earlier. The recombinant chicken interleukin-1β protein of the present invention helps to develop avian interleukin-1β vaccine adjuvant and uses in medical application and livestock production. Nowadays, all countries in the world including Taiwan have faced the problems of drug-resistance updates and new mutant recombinant virus including avian influenza; therefore, the recombinant chicken interleukin-1β protein of the present invention as a vaccine adjuvant can significantly enhance the effect of vaccine to protect livestock and poultry from disease threats.

Although the present invention has been described with reference to the preferred embodiments thereof, it is apparent to those skilled in the art that a variety of modifications and changes may be made without departing from the scope of the present invention which is intended to be defined by the appended claims.

What is claimed is:

1. A recombinant chicken interleukin-1β protein, which is encoded by the nucleotide sequence of SEQ ID NO:2.

2. The recombinant chicken interleukin-1β protein according to claim 1, which is used as an adjuvant to enhance the immunogenic potential of a vaccine for producing antibody one week earlier compared with a vaccine given only.

3. The recombinant chicken interleukin-1β protein according to claim 1, which is used as an adjuvant to enhance the immunogenic potential of a vaccine for retaining for at least four weeks compared with a vaccine given only.

4. The recombinant chicken interleukin-1β protein according to claim 1, which is a component of a vaccine.

5. The recombinant chicken interleukin-1β protein according to claim 4, wherein the vaccine is an inactivated vaccine or an attenuated vaccine.

6. The recombinant chicken interleukin-1β protein according to claim 1, which is a component of a vaccine adjuvant.

7. A method of producing antibody early and retaining for a longer period of time by using the recombinant chicken interleukin-1β protein according to claim 1.

8. The method according to claim 7, wherein a time for producing antibody is one week earlier.

9. The method according to claim 7, wherein the longer period of time is at least four weeks to extend the protective effect, and a booster is not given.

10. A method of producing antibody early and retaining for a longer period of time by using a vaccine supplemented with the recombinant chicken interleukin-1β protein according to claim 1.

11. The method according to claim 10, wherein a time for producing antibody is one week earlier.

12. The method according to claim 10, wherein the longer period of time is at least four weeks to extend the protective effect, and a booster is not given.
